(19) United States
(12) Patent Application Publication
(10) Pub. No.: US 2022/0395682 A1
(43) Pub. Date: Dec. 15, 2022

(54) OTOLOGIC SURGERY INSTRUMENT AND ROBOTIC INSTALLATION FOR CAPTURING AND HOLDING A COCHLEAR IMPLANT ELECTRODE HOLDER

(71) Applicant: COLLIN, BAGNEUX (FR)

(72) Inventor: Stéphane MAZALAIGUE, BAGNEUX (FR)

(21) Appl. No.: 17/770,923

(22) PCT Filed: Oct. 20, 2020
(86) PCT No.: PCT/FR2020/051893

§ 371 Date: Apr. 21, 2022

(30) Foreign Application Priority Data
Oct. 22, 2019 (FR) FR1911806

(51) Int. Cl. A61N 1/05

(52) U.S. Cl. CPC A61N 1/0541; A61B 34/30

(57) ABSTRACT

An otologic surgery instrument for inserting a cochlear implant electrode holder into a patient's inner ear, includes a gripping proximal end and a functional distal end for capturing and holding a portion of the electrode holder during the insertion. The proximal end includes a system for fastening to an articulated robot arm and the distal end includes a fixed member for gripping the portion of the electrode holder by clamping.

The present invention relates to an otologic surgery instrument for inserting a cochlear implant electrode holder into an inner ear of a patient. It also relates to a robotic installation comprising such an instrument.

The invention applies more particularly to an otologic surgery instrument of this type, comprising a proximal end for gripping and a functional distal end for capturing and holding a portion of the electrode holder during insertion.

A cochlear implant consists of an external part and an internal part, both of them made up of several components. The external part, intended to be worn on the ear and against the skin of the skull of an individual, generally comprises at least one microphone that captures a sound environment and transforms the sound into an electrical signal, a processor that filters the received information, in particular to process voice as a priority, and a transmitter by electromagnetic induction. The internal part comprises a receiver placed under the skin opposite the transmitter, electrically connected to an electrode holder intended to be placed in the tympanic duct of the individual's cochlea. The electrode holder is a soft and elastic component, usually made of silicone, which is difficult to insert into the inner ear during the surgical intervention required to install the inner part of the implant in a patient.

An example of an otologic surgical instrument for inserting a cochlear implant electrode holder into a patient's inner ear is described, for example, in EP 3 513 834 A1. It has a distal portion with a cannula in which the electrode holder is installed prior to surgery and a plunger for pushing the electrode holder during surgery for its extraction from the cannula and insertion into the patient's cochlea. It also has a cylindrical gripping proximal portion that can be directly grasped by a surgeon. Alternatively, this cylindrical proximal portion can be gripped in the hook of a robot arm although it is not specifically designed for this purpose. The instrument itself is quite complex to manufacture.

Another example of a robotic installation for inserting a cochlear implant electrode holder is described in U.S. Pat. No. 8,594,799 B2. The installation is illustrated in this document, but the instrument for capturing and gripping the electrode holder is not detailed. It is stated that a common insertion instrument can be used, without indicating which one and without really explaining how.

An example of a common instrument is a micro-clamp articulated at the distal end of a shaft, the proximal part of which is designed to be held in the hand. In order to free the surgeon's other hand, it is then necessary that the opening and closing of the micro-clamp be controlled by the hand holding the instrument. This results again in a complex instrument to manufacture. And for such an instrument to be carried by the articulated arm of a robot, the latter must be sufficiently sophisticated to not only hold the instrument but also operate its micro-clamp in opening and closing.

Thus, it may be desired to provide an otologic surgery instrument for inserting a cochlear implant electrode holder into an inner ear of a patient that avoids at least some of the above problems and constraints.

Thus, an instrument of this type is proposed comprising a gripping proximal end and a functional distal end for capturing and holding a portion of the electrode holder during insertion, wherein:
the proximal end comprises means for fastening to an articulated arm of a robot; and
the distal end comprises a member for gripping the portion of the electrode holder by clamping.

By having a proximal end specifically provided with means for fastening to an articulated robot arm, it is possible to free oneself from a constraint in terms of availability of the surgeon's hands and to provide a functional distal end for capture and holding that is much simpler than those envisaged in the prior art. Thus, a fixed gripping member by clamping, including in particular the fact that it is not articulated, is cleverly proposed in combination with the means for fastening to a robot arm to form a simple and inexpensive instrument to manufacture. It requires manual action by the surgeon to capture or release the desired electrode holder portion since it is fixed and in particular not articulated, but this is no longer a problem given its proximal end specifically intended to be held by a robot arm.

Optionally, the fixed gripping member is furthermore rigid, the gripping of the portion of the electrode holder being done by elasticity of the latter.

In this case, the otologic surgery instrument can be made of surgical stainless steel, in particular of 304L or 316L type according to AISI standard.

Also optionally, the fixed gripping member by clamping is a two-pronged fork extending along a distal portion of the instrument, for capturing and holding the portion of electrode holder between these two prongs.

In this case, the otologic surgery instrument may comprise several deviated portions all extending in one and the same plane parallel to a main plane of the two prongs of the fork.

Also in this case, the otologic surgery instrument may comprise several deviated portions all extending in one and the same plane perpendicular to a main plane of the two prongs of the fork.

Also optionally, each prong of the fork has a rectangular cross section.

Also optionally, the fixed gripping member by clamping is a gutter attached laterally to the free end of a distal portion of the instrument, for capturing and holding the portion of electrode holder within the interior volume of the gutter.

Also optionally, the means for fastening to the articulated arm of the robot comprise a locking groove hollowed longitudinally in the outer face of the proximal end of the instrument.

It is also proposed a robotic surgical intervention installation that includes:
a robot with an articulated arm that can be moved under electronic control; and
an otologic surgery instrument (30; 30) according to any of claims 1 to 9;
   wherein the articulated arm of the robot has complementary fastening means adapted to cooperate with the fastening means of the otologic surgery instrument.

The invention will be better understood with the aid of the following description, which is given solely by way of example and is made with reference to the appended drawings in which:

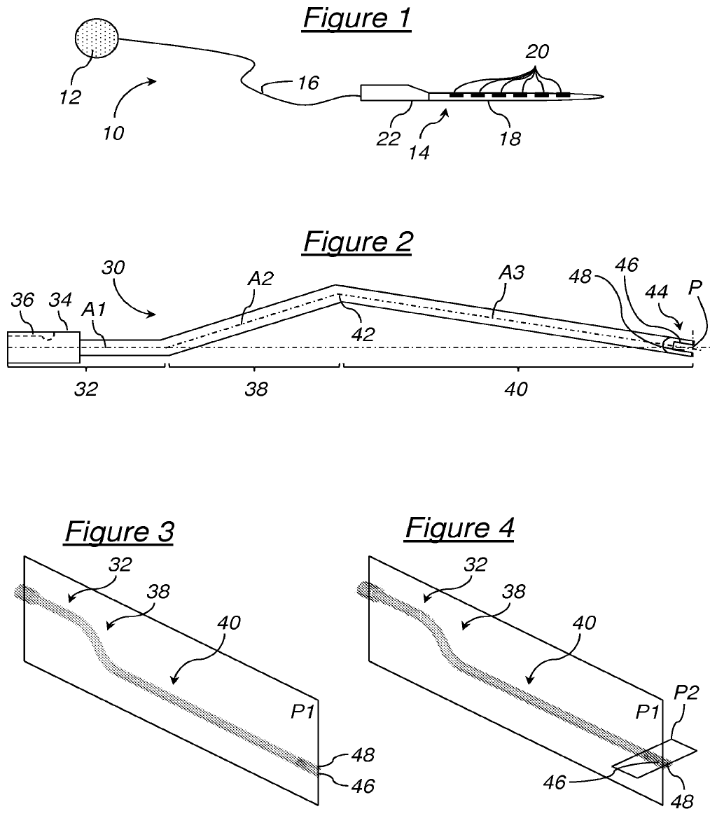

FIG. 1 diagrammatically represents the general structure of an example of an internal part of a cochlear implant with its electrode holder,

FIG. 2 represents diagrammatically and in longitudinal view the general structure of an otologic surgery instrument for the insertion of an electrode holder such as that of FIG. 1, according to a first embodiment of the invention

FIG. 3 illustrates the instrument of FIG. 2 in perspective,

FIG. 4 illustrates a variant of the instrument of FIG. 2 in perspective,

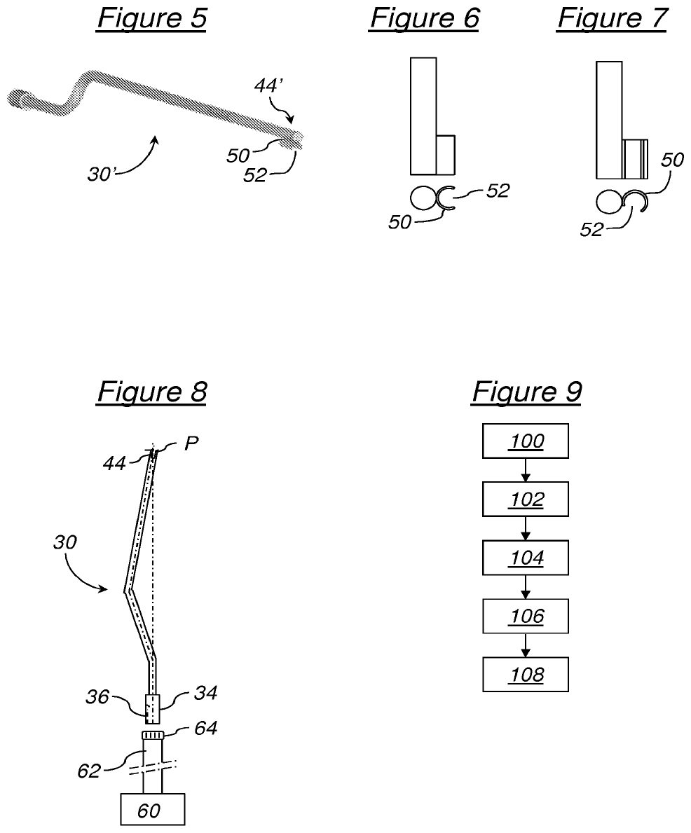

FIG. 5 represents diagrammatically and in perspective the general structure of an otologic surgery instrument for the insertion of an electrode holder such as that of FIG. 1, according to a second embodiment of the invention,

FIG. 6 is a set of two side and front views of a functional distal end of the device of FIG. 5,

FIG. 7 is a set of two side and front views of a variant for the functional distal end of the device of FIG. 5,

FIG. 8 represents diagrammatically and partially the general structure of a robotic surgical intervention installation including the instrument of FIG. 2, and

FIG. 9 illustrates the successive steps of a surgical intervention method using the robotic installation of FIG. 8.

The cochlear implant's inner portion 10 shown in FIG. 1 includes a receiver 12 connected to an electrode holder 14 via an electrical connection 16. The receiver 12 is intended to be placed under the skin of an individual opposite the transmitter of the outer portion not shown. The electrode holder 14 is intended to be placed in the tympanic duct of the individual's cochlea.

Various types of electrode holders exist on the market. The one shown in FIG. 1 has a main body 18 forming a support for a plurality of electrodes 20 as well as a proximal fin 22 facilitating its grip. The main body 18 is flexible and elastic, generally made of silicone, and is essentially cylindrical in shape with a circular cross section. The proximal fin 22 is optional, flexible, elastic and generally made of silicone like the main body 18, and extends the latter in a flat rectangular section.

The otologic surgical instrument 30 shown diagrammatically in FIG. 2 is adapted for insertion of the electrode holder of FIG. 1 into a patient's inner ear by gripping it by its proximal fin 22. In accordance with an advantageous but optional embodiment, it has several deviated portions to free the surgeon's aiming axis. Alternatively, it could be rectilinear.

It thus comprises a first holding or fastening portion 32, extending around a first rectilinear axis A1 and having a gripping proximal end 34 around this first rectilinear axis A1. The gripping proximal end 34 comprises means 36 for fastening to an articulated arm of a robot, for example materialized by a simple locking groove of semicircular section hollowed out longitudinally in its external face and ending in a partially spherical cavity of greater depth in conformity with the teaching of patent FR 2 998 344 B1.

The otologic surgery instrument 30 further includes a second deflection portion 38, integral with the first portion 32, extending without degrees of freedom from the first portion 32 in the principal direction of a second rectilinear axis A2 away from the first rectilinear axis A1. The angle between the axes A1 and A2 is, for example, between 2 and 10 degrees, preferably between 2 and 4 degrees, to give a very elongated shape to the instrument 30 with minimal lateral bulk.

The otologic surgery instrument 30 further includes a third functional support portion 40, integral with the second portion 38, extending without a degree of freedom from a distal end 42 of the second portion 38 about a third rectilinear axis A3 different from the second rectilinear axis A2 and approaching the first rectilinear axis A1. This third portion 40 has a functional distal end 44 which, in accordance with the present invention, includes a fixed member for gripping by clamping a desired portion of the electrode holder 14. The angle between axes A1 and A3 is, for example, between 2 and 10 degrees, preferably between 2 and 4 degrees. It should be sufficient to clear the field of vision of a user and facilitate the surgical gesture, while limiting the lateral bulk of the surgical instrument 30. Advantageously but optionally, in accordance with the general principles of patent application FR 3 066 378 A1, the third portion 40 is shaped, by its length and by the orientation of the third rectilinear axis A3, in such a way that the first rectilinear axis A1 about which the first portion 32 extends passes through the functional distal end 44—and more precisely through the fixed gripping member by clamping—of the third portion 40 at a pivot point P.

The three portions 32, 38 and 40 of the otologic surgery instrument 30 may be constituted, with the possible exception of the gripping proximal end 34 and the functional distal end 44, by one and the same rod (simple cylindrical rod of circular, square or rectangular section, or linkage) having two bends: the first bend at the junction of the first and second portions 32, 38; the second bend at the junction of the second and third portions 38, 40, i.e. at the distal end 42 of the second portion 38. In addition, all of the previously described component parts of the instrument 30 may be made of plastic, metal, stainless steel, titanium, or other biocompatible rigid materials or combinations of rigid materials suitable for surgical application in otorhinolaryngology. An example of a material particularly recommended for otologic surgery is surgical stainless-steel of 304L or 316L type according to the AISI (American Iron and Steel Institute) standard, which can constitute the entire instrument. In this case, the fixed gripping member by clamping forming the functional distal end 44 can be made of the same material as the rest of the rod forming the three deviated portions 32, 38 and 40. The advantage of having a fixed gripping member by clamping that is rigid like the rest of the instrument is the simplicity of manufacturing the assembly and its conformity to surgery requirements. The clamping of the desired portion of the electrode holder 14 can be done anyway thanks to the elasticity of the latter.

When the gripping proximal end 34 is an insert on the aforementioned rod, it may be formed as a substantially cylindrical sleeve including a bore along axis A1 for receiving the proximal end of the rod. Similarly, when the functional distal end 44 is an insert on the aforementioned rod, it may comprise a cylindrical projection for reception in a bore along the axis A3 at the distal portion of the rod. The advantage of a functional distal end 44 designed as an insert is that the rest of the instrument 30 can be manufactured in a standard way, as a “universal” instrument, and provided with a set of removable distal ends with different surgical functions other than at least that of gripping by clamping a portion of the electrode holder 14.

In terms of dimensions and conformations, the instrument 30 should generally have a total length (between the proximal end and the pivot point P) of between 10 and 20 cm, preferably between 10 and 15 cm, for use in otologic surgery. For example, when this length is close to 14 cm with an angle between the axes A1 and A3 of about 3.2 degrees, this makes it possible to shift the lateral observation distance by 1 cm, when the users eye is 18 cm from the pivot point P, i.e., about 4 cm longitudinally from the proximal end of the instrument 30 along the axis A1. This configuration proves satisfactory in use. Furthermore, a cylindrical rod diameter of between 1 and 2 mm is also suitable. It should be noted, however, that these values are given as an indication and that they could in particular be smaller or larger depending on the applications and contexts targeted.

According to the embodiment shown in FIG. 2, the fixed gripping member by clamping is more specifically a fork with two prongs 46 and 48 extending in the extension of the third portion 40 of the instrument 30, for capturing and holding the proximal fin 22 of the electrode holder 14 between these two prongs. The shape and dimensions of the two prongs 46, 48 are to be defined according to the shape and dimensions of the proximal fin 22 or any other portion of the electrode holder 14 that it is desired to capture and hold. In particular, in the case of a proximal fin of flat rectangular cross-section such as that shown in FIG. 1, parallel prongs of rectangular cross-section spaced to within a negative play depending on the thickness of the fin are appropriate. Alternatively, they could be of sections other than rectangular, in particular round, oval, etc.

The perspective view in FIG. 3 shows that the otologic surgery instrument 30, including its three deviated portions 32, 38, 40, extends in a plane P1 that is parallel to the main plane in which the two prongs 46 and 48 extend. It is even the same plane.

Alternatively, the perspective view of FIG. 4 shows that the otologic surgery instrument 30, including its three deviated portions 32, 38, 40, extends in a plane P1 that may be perpendicular to the main plane P2 in which the two prongs 46 and 48 extend in parallel. Other variants are also possible and depend mainly on the shape of the electrode holder to be captured and held as well as on the way in which it is desired to insert it into the patient's inner ear.

The otologic surgery instrument 30′ illustrated in FIG. 5 differs from those of FIGS. 2 to 4 by its functional distal end 44′ whose fixed gripping member by clamping is no longer a fork but a gutter 50, i.e., a hollow cylindrical element with a longitudinal opening 52 along its entire length, this gutter 50 being attached laterally against the free end of the third rectilinear portion of the instrument 30′. This allows the cylindrical main body 18 of the electrode holder 14 to be captured and held within the interior volume of the hollow, open cylinder formed by the gutter 50. This is particularly a preferred embodiment in the case where the electrode holder 14 does not include a proximal fin 22. The longitudinal opening is wide enough to easily capture the electrode holder 14 but also narrow enough to hold it during insertion into the patient's inner ear. In other words, the edges of the gutter 50 are advantageously folded toward each other to form an arc of a circle in cross-section slightly larger than the semicircle.

The side and front views of FIG. 6 show that the longitudinal opening 52 is opposite the third rod portion to which the gutter 50 is attached.

The side and front views in FIG. 7 show an alternative in which the longitudinal opening 52 is angularly offset by about 90 degrees to be positioned laterally of the third rod portion. Other alternatives are also possible and depend primarily on the shape of the electrode holder to be captured and held and how it is desired to be inserted into the patient's inner ear.

A robotic surgical intervention installation operating the otologic surgery instrument 30 is illustrated very diagrammatically in FIG. 8. It comprises a robot 60 provided with a carrying arm 62 movable under electronic control. An example of a robot compatible with an installation according to the invention is given in the article by Miroir et al, entitled “RobOtol: from design to evaluation of a robot for middle ear surgery”, published at the IEEE/RSJ International Conference on Intelligent Robots and Systems held from October 18 to 22, 2010 in Taipei (TW). It presents an architecture and kinematics particularly well suited to otologic surgeries of the middle or inner ear of patients, especially those concerning the insertion of the inner part of a cochlear implant.

The installation shown in FIG. 8 further comprises:
the otologic surgery instrument 30 whose gripping proximal end 34 is provided with the locking groove fastening means 36, and
complementary means for fastening the gripping proximal end 34 of the instrument 30 to the carrying arm 62 of the robot 60.

These complementary fastening means include, for example, a clamping ring 64 disposed at the free distal end of the carrying arm 62 of the robot 60, this carrying arm 62 being itself intended to receive by insertion the gripping proximal end 34. The locking groove 36 makes it possible to accompany and angularly guide the insertion of the instrument 30 into the carrying arm 62 of the robot 60, as taught in patent FR 2 998 344 B1. More generally, any known fastening means compatible with the configuration of the installation illustrated in FIG. 9 can be envisaged, in particular any means allowing the otologic surgery instrument 30 to be fastened in such a way that its main axis A1 corresponds with the intervention axis of the robot 60.

Obviously, the instrument 30 can be replaced by the instrument 30′ of FIG. 5.

FIG. 9 illustrates the successive steps of a surgical intervention method for a surgeon to insert the electrode holder 14 into a patient's cochlea using the robotic installation of FIG. 8.

In a first step 100, the surgeon fastens the otologic surgery instrument 30 or 30′ to the free end of the articulated arm 62 of the robot 60.

In a subsequent step 102, he/she grasps the electrode holder 14 and clamps the desired portion, proximal fin 22 or main body 18, into the fixed gripping member 44, 46 or 50.

During a step 104 of inserting the electrode holder 14 into the patient's inner ear, the surgeon precisely directs the electrode holder 14 into the cochlea by moving the functional distal end 44 or 44′ of the otologic surgery instrument 30 or 30′ carried by the articulated arm 62 of the robot 60 with the aid of a control peripheral (not shown). The surgical gesture is thus very precise and controlled.

When the electrode holder 14 is properly installed in the cochlea, the surgeon manually releases its portion clamped in the fixed gripping member 44, 46 or 50 in a step 106.

Finally, in a last step 108, the surgeon removes the otologic surgery instrument 30 or 30′ from the surgical area by precisely controlling its movement using the control device.

It clearly appears that a robotic installation such as the one described above can facilitate and secure a surgical procedure to insert a cochlear implant electrode holder into a patient's inner ear using an instrument that is simple to design, manufacture and use. The known gestures can be reproduced while eliminating tremors, involuntary movements and by ensuring a reproducibility of the surgical gesture.

It should also be noted that the invention is not limited to the above-described embodiments. Indeed, it will appear to those skilled in the art that various amendments can be made to the above-described embodiments, in the light of the teaching that has just been disclosed. In the detailed presentation of the invention made above, the terms used should not be interpreted as limiting the invention to the embodiments set forth in the present description, but should be interpreted to include all equivalents the anticipation of which is within the reach of those skilled in the art by applying their general knowledge to the implementation of the teaching just disclosed to them.

1. An otologic surgery instrument for inserting a cochlear implant electrode holder into a patient's inner ear, comprising a gripping proximal end and a functional distal end for capturing and holding a portion of the electrode holder during insertion, wherein:
the proximal end comprises means for fastening to an articulated arm of a robot; and
the distal end comprises a member for gripping the portion of the electrode holder by clamping;
wherein the member for gripping by clamping is fixed.

2. The otologic surgery instrument as claimed in claim 1, wherein the fixed gripping member is furthermore rigid, the gripping of the portion of the electrode holder being done by elasticity of the latter.

3. The otologic surgery instrument as claimed in claim 2, made of surgical stainless steel, in particular of 304L or 316L type according to AISI standard.

4. The otologic surgery instrument as claimed in claim 1, wherein the fixed gripping member by clamping is a two-pronged fork extending along a distal portion of the instrument, for capturing and holding the portion of electrode holder between these two prongs.

5. The otologic surgery instrument as claimed in claim 4, comprising several deviated portions all extending in one and the same plane parallel to a main plane of the two prongs of the fork.

6. The otologic surgery instrument as claimed in claim 4, comprising several deviated portions all extending in one and the same plane perpendicular to a main plane of the two prongs of the fork.

7. The otologic surgery instrument as claimed in claim 4, wherein each prong of the fork has a rectangular cross section.

8. The otologic surgery instrument as claimed in claim 1, wherein the fixed gripping member by clamping is a gutter attached laterally to the free end of a distal portion of the instrument, for capturing and holding the portion of electrode holder within the interior volume of the gutter.

9. The otologic surgery instrument as claimed in claim 1, wherein the means for fastening to the articulated arm of the robot comprise a locking groove hollowed longitudinally in the outer face of its proximal end.

10. A robotic surgical intervention installation comprising:
a robot with an articulated arm that is movable under electronic control; and
an otologic surgery instrument as claimed in claim 1;
wherein the articulated arm of the robot has complementary fastening means adapted to cooperate with the fastening means of the otologic surgery instrument.
